(19) United States
(12) Patent Application Publication
(10) Pub. No.: US 2009/0155595 A1
(43) Pub. Date: Jun. 18, 2009

(54) Polymeric Composites with a Hydrophilic Coating

(75) Inventor: William Lee, Revere, MA (US)

Correspondence Address: OCCHIUTI ROHLICEK & TSAO, LLP, 10 FAWCETT STREET, CAMBRIDGE, MA, 02138, US

(73) Assignee: eMembrane Inc., Providence, RI (US)

(21) Appl. No.: 11/954,808

(22) Filed: Dec. 12, 2007

(51) Int. Cl. B32B 27/00; B32B 27/34; B32B 27/36; B32B 27/40; B32B 27/30; A61F 2/16

(52) U.S. Cl. 428/412; 428/411.1; 428/422; 428/474.4; 428/479.3; 428/421; 428/423.1; 428/480; 606/107

(57) ABSTRACT

A polymeric composite including (1) a substrate formed of a moldable polymer; (2) a first polymeric layer containing a base polymer, the first layer adhering to a surface of the substrate by physical entrapment of at least some molecules of the base polymer in the substrate; and (3) a second polymeric layer containing a hydrophilic polymer, the second layer adhering to a surface of the first layer by physical entrapment of at least some molecules of the hydrophilic polymer in the first layer. Also disclosed is a device including such a composite for delivering an intraocular lens.

### BACKGROUND OF THE INVENTION

Synthetic polymers are widely used nowadays to fabricate myriads of products, including medical devices. Many such medical devices, e.g., intraocular lens injector tubes, require a hydrophilic surface. See, e.g., U.S. Pat. No. 5,803,925. Yet, polymeric materials in general are relatively hydrophobic.

A number of methods have been developed for applying a durable hydrophilic coating on polymeric substrates in recent years. However, there remains a need for simpler and less expensive coating processes.

### SUMMARY OF THE INVENTION

One aspect of this invention relates to a facile method for coating a polymeric substrate with a hydrophilic polymer.

The method includes (1) applying a base polymer dispersed in a first solvent onto a surface of a substrate formed of a moldable polymer, the first solvent being capable of penetrating into the substrate; (2) removing the first solvent to leave behind on the surface a first polymeric layer formed of the base polymer, at least some molecules of which are partially entrapped in the substrate; (3) applying a hydrophilic polymer dispersed in a second solvent onto the first layer, the second solvent being capable of penetrating into the first layer; and (4) removing the second solvent to leave behind on the first layer a second polymeric layer formed of the hydrophilic polymer, at least some molecules of which are partially entrapped in the first layer, thereby producing a substrate with a hydrophilic surface. More specifically, the substrate is coated with the first layer (i.e., as an inner base coating) adhering to the substrate by physical entrapment (i.e., not by covalent bonding), and the first layer is in turn coated with the second layer (i.e., as an outer hydrophilic coating) adhering to the first layer also by physical entrapment.

Moldable polymers that can be used to prepare the substrate include but are not limited to polypropylene, polycarbonate, polyethylene, acryl-butadienestyrene, polyamide, polychlorotrifluoroethylene, polytetrafluoroethylene, polyvinyl chloride, polyvinyldene fluoride, ethylene tetrafluoroethylene, ethylene chlortrifluoroethylene, perfluoroalkoxy, styrene, polymethylpentene, polymethylmetyacrylate, polystyrene, polyetheretherketone, and tetrafluoroethylene. Among them, polypropylene and polycarbonate are preferred. For a substrate made from one of these two moldable polymers, the first solvent mentioned above for both dispersing the base polymer and penetrating the substrate can be acetaldehyde, hydrochloric acid, sulfuric acid, benzene, ether, tetrahydrofuran, toluene, methanol, ethanol, propanol (including isopropyl alcohol), butanol, dimethylacetamie, xylene, or a combination thereof. For a substrate molded from polypropylene, solvents such as hydrofluoric acid, ammonium hydroxide, chlorobenzene, hexane, and phenol can also be used. For a substrate molded from polycarbonate, solvents such as acetone, acetonitrile, and cyclohexane can also be used. The second solvent mentioned above for both dispersing the hydrophilic polymer and penetrating the first layer (i.e., the base coating on the substrate) can be selected based on the types of hydrophilic polymer and base polymer used in this method. For example, when polyvinylpyrrolidone is used as the hydrophilic polymer and polyurethane is used as the base polymer, the second solvent can be a mixture of tetrahydrofuran and ethanol.

Suitable base polymers for use in this method include but are not limited to polyurethane, polyacrylate, polymethacrylate, polyvinyl chloride, polyamide, and polyester/alkyd copolymer.

Suitable hydrophilic polymers for use in this method include but are not limited to polyvinylpyrrolidone, poly-N-vinyl lactams, poly(ethylene oxide), poly(propylene oxide), polyethylene glycol, polyvinyl pyridine, polysaccharides, polycarboxyl methyl cellulose, polypeptides, polyhydroxyethyl methacrylate, poly sodium styrene sulfonate, heparin, polyacrylamides, cellulosic (e.g., methyl cellulose), polyacrylic acid, and polyvinyl ester.

Another aspect of this invention is a polymeric substrate having a hydrophilic surface prepared by the above-described method. Thus, also within the scope of this invention is a polymeric composite including a substrate formed of a moldable polymer; a first polymeric layer containing a base polymer, the first layer adhering to a surface of the substrate by physical entrapment of at least some molecules of the base polymer in the substrate; and a second polymeric layer containing a hydrophilic polymer, the second layer adhering to a surface of the first layer by physical entrapment of at least some molecules of the hydrophilic polymer in the first layer.

An embodiment of the above-described polymeric composite can be part of a device for receiving and delivering an intraocular lens into an eye. More specifically, the device includes a tapered tube formed of a moldable polymer; a first polymeric layer, including a base polymer, coated on the inner surface of the tube by physical entrapment of at least some molecules of the base polymer in the tube; and a second polymeric layer, including a hydrophilic polymer, coated on the first polymeric layer by physical entrapment of at least some molecules of the hydrophilic polymer in the first polymeric layer. Given the hydrophilic inner surface of the tube, an intraocular lens placed in it can be easily pushed, by a plunger configured to enter the tube from the wide end, through the tapered end into the eye.

The hydrophilic coating method of this invention is simple, inexpensive, and reliable, as it is based on an unexpected finding that a durable hydrophilic polymeric layer can be formed on a base polymer layer pre-coated on a polymeric substrate without relying on covalent bonding among the two layers and the substrate.

Other advantages or features of the present invention will be apparent from the following detailed description of several embodiments, and also from the appending claims.

### DETAILED DESCRIPTION OF THE INVENTION

Polypropylenes and polycarbonate are preferred moldable polymers for forming substrates for use in the present invention in view of their low cost, inert property, and well-studied behavior in molding and processing. Many other polymers, such as polyamide, cellulose acetate, and acrylic polymer or copolymer, can also be molded into substrates.

To practice the method of this invention, a substrate is first coated with a base polymer to form a first layer, which is in turn coated with a hydrophilic polymer to form a second layer.

A base polymer is a durable polymer that does not cause any reaction with the substrate on which it is coated and enhances the physical integrity of the hydrophilic layer coated on it. Examples of a base polymer include polyurethane and polyvinyl chloride. To coat a substrate, a base polymer is first dispersed (i.e., dissolved or suspended) in a solvent (e.g., a pure solvent or a mixture of two solvents) that is capable of penetrating the substrate on which the base polymer is to be coated. In other words, the solvent, in addition to dispersing the base polymer, also plays the role of volumetric penetrating and swelling the substrate. Note that less chemical compatibility between the solvent and the substrate (i.e., more effect of the solvent on the substrate) leads to more penetration of the solvent into, and more swelling of, the substrate. When the substrate is swollen, the base polymer in the solvent diffuses and penetrates into the substrate during the coating process. In general, a base polymer is coated on a substrate as follows. A base polymer-containing solvent is applied to a surface of the substrate by dipping, spraying, brushing, or using a pipette and any other suitable method. The solvent is then removed by, e.g., heating, air drying, or vacuuming. Removal of the solvent results in formation on the surface of the substrate a layer of the base polymer, at least some molecules of which are physically entrapped inside the substrate.

A hydrophilic polymer is a polymer which swells in the presence of water to provide a lubricious surface. Examples of a hydrophilic polymer include polyvinylpyrrolidone and poly(ethylene oxide). A hydrophilic polymer, when hydrated, possesses relatively less physical integrity because of the high water content. The method of this invention allows the formation of an interpenetrating polymer network in which a hydrophilic polymer and a base polymer interact with each other such that the hydrophilic polymer is physically entrapped by the base polymer and, as a result, its loss to the environment is minimized when wet. Such an interpenetrating polymer network can be formed by coating a base polymer layer with a hydrophilic polymer in a manner analogous to that in which a surface of a substrate is coated with a base polymer layer as described in the preceding paragraph. A hydrophilic polymer layer may also contain a base polymer that is either the same as or different from that in the base polymer layer onto which it is coated. Such a layer can be formed by using a solvent containing both a hydrophilic polymer and a base polymer.

The thickness of the base polymer layer or the hydrophilic polymer layer can be controlled by the viscosity of the coating solution and the duration of the coating process. In general, higher viscosity and longer coating time result in a thicker coating layer. However, a coating thickness optimization step is necessary for each specific application. Note that the durability of the hydrophilic polymer layer reflects the strength of the interpenetrating polymer network, i.e., the adhesion of the base polymer layer to the substrate and the adhesion of the hydrophilic polymer layer to the base polymer layer. This strength depends on the degree of the physical entanglement (interpenetration), which can be derived from the thickness of each layer. The molecular weight of the polymer is another factor that determines the durability of the hydrophilic polymer layer; namely, use of a polymer of a higher molecular weight leads to more physical entanglement and thus higher durability. The durability of the hydrophilic polymer layer can be qualitatively determined by retention of the slippery feel when wet or when rubbed. Other durability tests include, but are not limited to, repeated measurements of friction and measurements of lubricity before and after autoclaving. In testing coated intraocular lens injectors, the presence of more coating residue on the delivered lens is indicative of poorer durability of the hydrophilic polymer layer.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever. Note that, in Example 1, plasma was used to clean polypropylene slides to be coated and, as a result, covalent boding might form between the slides and the base coating applied onto them. Such covalent bonding, if any, is not within the meaning of the term “covalent bonding” or “covalent bonded” as used in this disclosure. All of the three US patents cited herein are incorporated in their entirety by reference.

### EXAMPLE 1

A number of polypropylene slides were coated with a hydrophilic layer following the procedures described below.

To clean the slides, they were first sonicated in isopropyl alcohol for 2 minutes, dried with an air gun, and then treated with O₂/Argon plasma at 150 watts, 250 mTorr for 5 minutes.

The slides thus cleaned were base coated as follows. Each was submerged in one of the following two base coating solutions: 5% by weight ChronoThane H (an aromatic ether based polyurethane purchased from CardioTech International, Inc., Woburn, Mass.) in tetrahydrofuran (THF) and 10% by weight ChronoThane™ H also in THF. After 20 minutes, the slides were removed from the base coating solutions and cured in an oven at 65° C. for 1.5 hours.

The base-coated slides were then top coated as follows. They were dipped into a top coating solution, i.e., 5% by weight polyvinylpyrrolidone (PVP) in THF/ethanol (weight ratio=8.5:1), removed quickly, and cured in an oven at 65° C. for 12 hours.

All of the dual-coated slides were evaluated for their lubricity and durability. Lubricity was determined by both (1) feeling of finger touching and (2) wiping with bare fingers and deionized water. Durability was determined by comparing lubricity (1) before and after sonicating the sample in deionized water for 5 minutes and/or (2) before and after submerging the sample in deionized water overnight.

The results indicate that the slides base coated with 5% ChronoThane H was somewhat more lubricious than those coated with 10% ChronoThane H. The former slides were also more durable. Unexpectedly, both their lubricity and durability, despite absence of covalent bonding between the base and top coatings, were comparable to those of slides dual-coated by the same procedures except that a crosslinker-containing top coating solution, i.e., polyurethane, PVP, and aziridine in water (weight ratio=18.49:10.41:0.36:21.09) was used so that covalent bonding between the two coatings formed and the top coating was cured for 4 hours. Similar crosslinker-containing coating solutions are described in U.S. Pat. Nos. 6,238,799 and 6,866,936.

### EXAMPLE 2

Slides were dual-coated following the procedures described in Example 1 above except that a different top coating solution, i.e., PVP and ChronoThane H in THF/ethanol (weight ratio=0.49:0.49:44.93:4.08), was used.

The slides thus coated were then subjected to lubricity and durability tests also described in Example 1. All of them exhibited both acceptable lubricity and acceptable durability.

### EXAMPLE 3

Intraocular lens (IOL) injectors molded from polypropylene were dual-coated as follows.

A base coating solution, i.e., 10% by weight ChronoThane H in THF, was placed inside the IOL-receiving chamber of an IOL injector with a disposable pipette. The solution was allowed to stay in the chamber for about 20 minutes. After removal of the excess coating solution using a TechniCloth wiper (ITW Texwipe, Mahwah, N.J.), the base coating was cured in an oven at 65° C. for 1.5 hours.

A top coating solution, i.e., 5% by weight PVP in THF/ethanol (weight ratio=8.5:1), was placed inside the base coated chamber and the excess solution removed from it in the same manner. The top coating thus formed was evened out using an air gun before it was cured in an oven at 65° C. for 12 hours.

Each of the dual-coated IOL injectors was tested with an IOL. In all cases, the IOL passed through the chamber with little resistance.

### EXAMPLE 4

IOL injectors molded from polypropylene were dual-coated in the same manner as that described in Example 3 above except that a different top coating solution, i.e., PVP and ChronoThane H in THF/ethanol (weight ratio=0.49:0.49:44.93:4.08), was used.

The IOL injectors thus obtained were tested for the ease in which an IOL passed through the chamber. They exhibited even greater lubricity than those prepared in Example 3.

### Other Embodiments

From the above description, one skilled in the art can easily ascertain the essential characteristics of the present invention, and without departing from the spirit and scope thereof, can make various changes and modifications of the invention to adapt it to various usages and conditions. For example, while in a polymeric composite of this invention no covalent bonding is formed among the substrate, base polymer layer, and hydrophilic polymer layer, molecules of the base polymer can be covalently bonded to each other. To make such an embodiment, a base polymer having functional moieties capable of undergoing crosslinking reaction is used. Other embodiments are also within the claims.

What is claimed is:

1. A polymeric composite comprising:
a substrate formed of a moldable polymer;
a first polymeric layer including a base polymer, the first layer adhering to a surface of the substrate by physical entrapment of at least some molecules of the base polymer in the substrate; and
a second polymeric layer including a hydrophilic polymer, the second layer adhering to a surface of the first layer by physical entrapment of at least some molecules of the hydrophilic polymer in the first layer.

2. The method of claim 1, wherein the moldable polymer is polypropylene, polycarbonate, polyethylene, acryl-butadienestyrene, polyamide, polychlorotrifluoroethylene, polytetrafluoroethylene, polyvinyl chloride, polyvinyldene fluoride, ethylene tetrafluoroethylene, ethylene chlortrifluoroethylene, perfluoroalkoxy, styrene, polymethylpentene, polymethylmetyacrylate, polystyrene, polyetheretherketone, or tetrafluoroethylene.

3. The composite of claim 2, wherein the moldable polymer is polypropylene or polycarbonate.

4. The composite of claim 1, wherein the base polymer is polyurethane, polyacrylate, polymethacrylate, polyvinyl chloride, polyamide, or a polyester/alkyd copolymer.

5. The composite of claim 4, wherein the base polymer is polyurethane.

6. The composite of claim 1, wherein the hydrophilic polymer is polyvinylpyrrolidone, poly-N-vinyl lactams, poly(ethylene oxide), poly(propylene oxide), polyethylene glycol, polyvinyl pyridine, polysaccharides, polycarboxyl methyl cellulose, polypeptides, polyhydroxyethyl methacrylate, poly sodium styrene sulfonate, heparin, polyacrylamides, cellulosic, polyacrylic acid, or polyvinyl ester.

7. The composite of claim 6, wherein the hydrophilic polymer is polyvinylpyrrolidone.

8. The composite of claim 2, wherein the base polymer is polyurethane, polyacrylate, polymethacrylate, polyvinyl chloride, polyamide, or a polyester and alkyd copolymer.

9. The composite of claim 4, wherein the hydrophilic polymer is polyvinylpyrrolidone, poly-N-vinyl lactams, poly(ethylene oxide), poly(propylene oxide), polyethylene glycol, polyvinyl pyridine, polysaccharides, polycarboxyl methyl cellulose, polypeptides, polyhydroxyethyl methacrylate, poly sodium styrene sulfonate, heparin, polyacrylamides, cellulosic, polyacrylic acid, or polyvinyl ester.

10. The composite of claim 6, wherein the moldable polymer is polypropylene, polycarbonate, polyethylene, acryl-butadienestyrene, polyamide, polychlorotrifluoroethylene, polytetrafluoroethylene, polyvinyl chloride, polyvinyldene fluoride, ethylene tetrafluoroethylene, ethylene chlortrifluoroethylene, perfluoroalkoxy, styrene, polymethylpentene, polymethylmetyacrylate, polystyrene, polyetheretherketone, or tetrafluoroethylene.

11. The composite of claim 10, wherein the base polymer is polyurethane, polyacrylate, polymethacrylate, polyvinyl chloride, polyamide, or polyester/alkyd copolymer.

12. The composite of claim 1, wherein the moldable polymer is polypropylene, the base polymer is polyurethane, and the hydrophilic polymer is polyvinylpyrrolidone.

13. A device for delivering an intraocular lens into an eye, said device comprising:
a tapered tube formed of a moldable polymer, the tube having a tube inner surface;
a first polymeric layer including a base polymer, the first polymeric layer having an first polymeric outer surface and a first polymeric inner surface, the first polymeric outer surface adhering to the tube inner surface by a physical entrapment of at least some molecules of the base polymer in the tube; and
a second polymeric layer including a hydrophilic polymer, the second polymeric layer having a second polymeric outer surface adhering to the first polymeric inner surface by physical entrapment of at least some molecules of the hydrophilic polymer in the first polymeric layer.

14. The device of claim 13, wherein the moldable polymer is polypropylene, polycarbonate, polyethylene, acryl-butadienestyrene, polyamide, polychlorotrifluoroethylene, polytetrafluoroethylene, polyvinyl chloride, polyvinyldene fluoride, ethylene tetrafluoroethylene, ethylene chlortrifluoroethylene, perfluoroalkoxy, styrene, polymethylpentene, polymethylmetyacrylate, polystyrene, polyetheretherketone, or tetrafluoroethylene.

15. The device of claim 14, wherein the moldable polymer is polypropylene or polycarbonate.

16. The device of claim 13, wherein the base polymer is polyurethane, polyacrylate, polymethacrylate, polyvinyl chloride, polyamide, or a polyester/alkyd copolymer.

17. The device of claim 16, wherein the base polymer is polyurethane.

18. The device of claim 13, wherein the hydrophilic polymer is polyvinylpyrrolidone, poly-N-vinyl lactams, poly(ethylene oxide), poly(propylene oxide), polyethylene glycol, polyvinyl pyridine, polysaccharides, polycarboxyl methyl cellulose, polypeptides, polyhydroxyethyl methacrylate, poly sodium styrene sulfonate, heparin, polyacrylamides, cellulosic, polyacrylic acid, or polyvinyl ester.

19. The device of claim 18, wherein the hydrophilic polymer is polyvinylpyrrolidone.

20. The device of claim 14, wherein the base polymer is polyurethane, polyacrylate, polymethacrylate, polyvinyl chloride, polyamide, or a polyester and alkyd copolymer.

21. The device of claim 16, wherein the hydrophilic polymer is polyvinylpyrrolidone, poly-N-vinyl lactams, poly(ethylene oxide), poly(propylene oxide), polyethylene glycol, polyvinyl pyridine, polysaccharides, polycarboxyl methyl cellulose, polypeptides, polyhydroxyethyl methacrylate, poly sodium styrene sulfonate, heparin, polyacrylamides, cellulosic, polyacrylic acid, or polyvinyl ester.

22. The device of claim 18, wherein the moldable polymer is polypropylene, polycarbonate, polyethylene, acryl-butadienestyrene, polyamide, polychlorotrifluoroethylene, polytetrafluoroethylene, polyvinyl chloride, polyvinyldene fluoride, ethylene tetrafluoroethylene, ethylene chlortrifluoroethylene, perfluoroalkoxy, styrene, polymethylpentene, polymethylmetyacrylate, polystyrene, polyetheretherketone, or tetrafluoroethylene.

23. The device of claim 22, wherein the base polymer is polyurethane, polyacrylate, polymethacrylate, polyvinyl chloride, polyamide, or polyester/alkyd copolymer.

24. The device of claim 13, wherein the moldable polymer is polypropylene, the base polymer is polyurethane, and the hydrophilic polymer is polyvinylpyrrolidone.

25. The device of claim 13, wherein the second polymeric layer also includes a base polymer.
